(19) United States
(12) Patent Application Publication
(10) Pub. No.: US 2005/0118274 A1
(43) Pub. Date: Jun. 2, 2005

(54) Method and device for feeding living cells into a biological body fluid

(76) Inventor: Augustinus Bader, Parthenstein-Klinga (DE)

Correspondence Address: DAVIS & BUJOLD, P.L.L.C., FOURTH FLOOR, 500 N. COMMERCIAL STREET, MANCHESTER, NH, 03101-1151, US

(21) Appl. No.: 10/502,482

(22) PCT Filed: Jan. 22, 2003
(86) PCT No.: PCT/EP03/00579

(30) Foreign Application Priority Data
Jan. 26, 2002 (DE) 102 02 982.2

(51) Int. Cl. A61K 38/46; A61K 9/16; A61K 45/00; A61K 31/557

(52) U.S. Cl. 424/490; 424/93.7; 424/94.65; 514/573

(57) ABSTRACT

Disclosed is a method for feeding living cells into a body fluid, particularly into a blood stream, according to which one or several of the cells are combined into capsule units by an enveloping substance surrounding the cells. In order to prevent coagulation or rejection, coagulation-inhibiting agent or coagulation-preventing structures are placed in or on the enveloping substance.

The invention relates to a method and a device for feeding living cells into a biological body fluid, particularly into a blood stream, according to which method one or more of the cells are combined into capsule units by means of an enveloping substance which surrounds the cells.

The invention also relates to a device for carrying out the method and to capsule units for this purpose.

It is known, for example in the event of liver failure, to connect the patient to a liver reactor. To do so, however, it is necessary to perform plasma separation to separate the cellular components of the blood, in particular the red blood cells, from the actual plasma in which the fibrin is still contained. Passing the blood directly through the liver reactor, in which liver cells are encapsulated, i.e. in which a plurality of cells are combined into larger capsule units by means of an enveloping substance which surrounds the cells, is not possible because the blood would coagulate in the liver reactor. For this reason, a plasma separator would have to be interposed in which the plasma is separated from the blood. However, a disadvantage of this would be that the capillaries present in the plasma separator would clog up relatively quickly, with the result that the separator would become ineffective and would have to be replaced at considerable effort and cost. This would generally happen after just eight to ten hours. A much more serious disadvantage, however, would be that the plasma separation or blood separation would lead to hemolysis, which would be traumatic for the patient and, in the case of prolonged treatment, could even be fatal. In previous bioreactors, the biological systems are either exposed to plasma directly and unprotected and thus trigger coagulation and cause complement activation, or are positioned behind a plasma-separating membrane. A serious disadvantage of the plasma separators employed is also that so-called membrane fouling takes place, i.e. clogging caused by deposits on the pores. For this reason, these systems have to be replaced in the short term.

It is known that a damaged liver is able to regenerate as long as healthy liver cells are still present. Therefore, if it were possible to connect a patient to a liver reactor for a longer period, a damaged liver, for example damaged by poisoning or by a tumor operation, would have the chance to regenerate if given sufficient time. However, several days are needed for this, which fact leads to the aforementioned problems.

The object of the present invention is therefore to make available a method and a device with which body fluid, for example blood, can be treated directly, without the disadvantages of plasma separation.

According to the invention, this object is achieved by a method for feeding living cells into a body fluid, in which method a plurality of cells surrounded by an enveloping substance are combined into capsule units, and, in order to prevent coagulation or rejection, coagulation-inhibiting agents or coagulation-preventing structures are placed in or on the enveloping substance.

A device for carrying out this method is provided with a container with an inlet for the body fluid, for example blood, and with an outlet for blood, a multiplicity of capsule units being arranged in the container, which capsule units are each formed by a plurality of cells surrounded by an enveloping substance, said enveloping substance being provided with coagulation-inhibiting agents or coagulation-preventing structures.

According to the invention, coagulation-inhibiting agents or coagulation-preventing structures are now added to the cells or to the capsule units with the cells arranged in them. In this way, for example, a blood stream can be conveyed directly through the “bioreactor” created in this way, without separation by technical membranes, such as, for example, capillaries in hollow-fiber bioreactors. This means there is no need for preliminary plasma separation, with its resulting disadvantages.

According to the invention, the blood obtained directly from a patient can now be passed through the bioreactor without coagulation of the blood taking place. After it has been cleaned, the blood can then be returned directly to the patient.

One of the most important differences from the prior art is that this method can extend over several days, which means, for example, that patients with liver damage thus have the possibility of having their liver regenerate itself.

A further advantage of the method according to the invention and of the device is that, after cell recovery and the formation of capsule units and suitable pre-cultivation, the capsule units, which for example are introduced into a bag-like container as bioreactor, can be frozen. This affords the possibility of being able to keep such bioreactors in store and of being able to use them immediately on the patient, as and when required, after thawing.

Suitable agents for measures by which blood coagulation can be prevented are, for example, active coagulation-inhibiting agents such as heparins, hirudins or prostaglandins and thromboxane structures.

Another solution here is for passive coagulation-preventing structures to be built into the capsule units or attached to them. Passive coagulation-preventing structures avoid a situation where, for example, the red blood cells in the blood stream recognize the capsule units as foreign bodies and trigger coagulation of the blood. In practice, the red blood cells are in this way presented with what appears to be a natural cell membrane surface. Lipids, for example, are suitable for this purpose, for example phosphatidylcholine, with proteins or with glycoproteins. Similarly, glycocalyx structures, produced synthetically or obtained pre-operatively from erythrocyte surfaces, can be integrated in or on the surfaces of the hybrid capsules, matrix. (fibrin, collagen, albumin). They thus form a pattern of hydrophilic and hydrophobic groups which in particular avoid the adhesion of other cells and coagulation of the blood. The biological components can also be replaced by synthetic components, for example hydrophilic and hydrophobic polymers.

As will be evident, the cells arranged in the capsule units are in this way cultivated directly in the blood stream which is guided through the bioreactor. In addition to the advantages already mentioned, this also provides a considerable cost reduction. Oxygenation of the bioreactor during in vivo use is not necessary, because the blood with red blood cells passing through the bioreactor means of course that oxygen is introduced directly into the system.

In practice, a patient can be treated with the bioreactor according to the invention in the same way as, for example, in blood transfusion ox kidney dialysis.

Polymers can be used as enveloping substances for forming capsule units containing a plurality of cells. Examples of suitable polymers are hydrogels, such as alginates, collagens, chitins, and albumins and gels. In the same way, however, biological polymers can also be used, for example polylactide. A preferred area of application of the bioreactor is the cultivation of liver cells and kidney cells or other cells in the blood stream.

The method described above relates to an extracorporeal use which comes only temporarily into contact with body fluids, for example blood. However, the method according to the invention can also be used for intracorporeal systems which in most cases are intended to remain in the body throughout life or for as long as possible and can be given the coating according to the invention in order to prevent stopping forces.

Examples are artificial biological vessels, implants, e.g. heart valves and vein valves. These are technical or biological structures whose surfaces are colonized with different cells. These also include, for example, fibroblasts, smooth muscle cells and endothelial cells. In cases where endothelialization is incomplete (particularly in short or incomplete colonization processes), exposure of the underlying matrix can easily occur. The matrix can consist of collagens, fibrin or synthetic polymers.

To avoid immediate triggering of coagulation, the same factors and constituents as in the microcapsules can be integrated in such hybrid structures in or over the matrix.

A further conceivable area of application is, for example, for diabetic patients. In this case, it is conceivable for a bioreactor with cells which produce insulin to be fitted as an implant into a patient's body. Because of the coagulation-inhibiting agents or coagulation-preventing structures according to the invention, the body does not then regard this bioreactor as a foreign body and does not reject it. Using suitable endogenous islet cells which are produced from embryonal stem cells or from cord blood or marrow cells and are introduced into the capsule units and then injected directly into the patient's blood stream, it would be possible, at various sites in the organism, to form small glucose sensors which release sutable amounts of insulin when the glucose level rises.

Examples of possible bioreactors are hollow-fiber bioreactors, solid-bed reactors, stirred fermenters, microcarrier systems, and flat-membrane bioreactors.

Instead of blood as the body fluid, the method according to the invention can also be used, for example, with plasma or cerebrospinal fluid.

An illustrative embodiment of the invention is described below with reference to the drawing, in which:

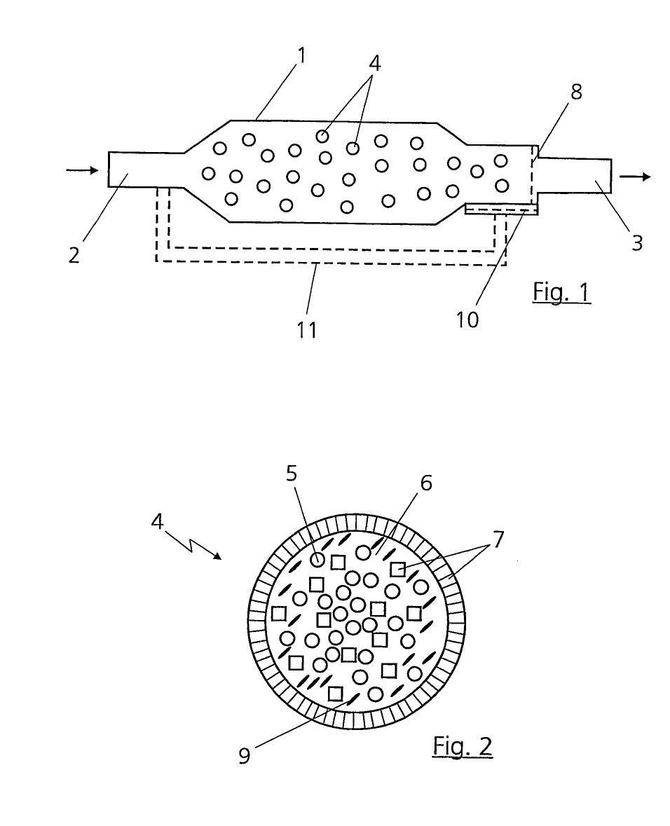

FIG. 1 shows a device according to the invention; and

FIG. 2 shows a greatly enlarged representation of a capsule unit.

FIG. 1 shows diagrammatically a container 1, which can be in the form of a bag for example, with an inlet 2 for blood to be purified, and with an outlet 3 for the purified blood. Arranged inside the container 1 there are a large number of capsule units 4 in each of which a plurality of cells 5, for example liver cells (e.g. about 4 to 10 cell layers), are arranged. The cells 5 are surrounded by an enveloping substance 6 and in this way form for example a spherical capsule unit 4. The connection between the enveloping structure and the cells 5 can be effected, for example, by appropriate mixing. Coagulation-inhibiting agents or coagulation-preventing structures 7 are also subsequently introduced into the mixture. When using coagulation-inhibiting agents, for example heparin, the coagulation-inhibiting agent will be mixed with the enveloping substance 6 so that the coagulation-inhibiting agents are arranged not only on the surface, but also in the inside. By virtue of the porous configuration of the capsule units 4 and enveloping substance 6, the blood also flows through the capsule itself.

When using coagulation-preventing structures 7, care will be taken to ensure that these structures are preferably arranged on the surface of the capsule unit 4 or in the outer area thereof, so that the capsule unit 4 is not identified as a foreign body by the red blood cells. Lipids, for example, can be used as coagulation-preventing structures 7.

To ensure that the capsule units 4 are not washed out of the container 1 during detoxification of the blood or cultivation of the cells 5, the outlet 3 has to be made suitably small. Alternatively, it is also possible to provide a filter arrangement 8 in front of the outlet. A further possibility for retaining capsule units 4 in the container 1 can be to use a “magnet trap”. For this purpose, the capsule units 4 will be additionally provided with tiny magnet parts or magnetizable substances 9, with the result that the capsule units 4 react on a magnetic separator 10 (see broken lines in FIG. 1) with a magnet arranged in the outlet area of the container 1. Here too, the magnetizable substances 9 will preferably be arranged on the surface or in the outer area of the capsule units 4.

The capsule units 4 are then held back by the magnetic separator arrangement 10 and can be fed via a line 11 (shown by broken lines) back to the inlet area of the container 1. There is in principle no great problem in separating the capsule units 4 from the blood in order to avoid the capsule units 4 being entrained with the blood stream. This is because red blood cells have a size of ca. 7 to 10 μm, whereas the capsule units are generally given a size or diameter of 50 to 100 μm or 200 μm.

1-25. (canceled)

26. A method for feeding living cells into a biological body fluid, particularly into a blood stream, in which method one or more cells (5) surrounded by an enveloping substance (6) are combined into capsule units (4), and, in order to prevent coagulation or rejection, coagulation-inhibiting agents or coagulation-preventing structures (7) are placed in or on the enveloping substance (6).

27. The method according to claim 26, further comprising the step of using heparins as the coagulation-inhibiting agents (7).

28. The method according to claim 26, further comprising the step of using hirudins as the coagulation-inhibiting agents (7).

29. The method according to claim 26, further comprising the step of using prostagladins as the coagulation-inhibiting agents (7).

30. The method according to claim 26, further comprising the step of using thromboxane structures as the coagulation-inhibiting agents (7).

31. The method according to claim 26, further comprising the step of using a polymer as the enveloping substance (6).

32. The method according to claim 31, further comprising the step of using a synthetic polymer as the enveloping substance (6).

33. The method according to claim 32, further comprising the step of using polylactide, polyurethane, polyester, gels, hydrogels or silicone as the synthetic polymer.

34. The method according to claim 31, further comprising the step of using biological polymers.

35. The method according to claim 34, further comprising the step of using alginates, collagens or chitins as the biological polymers.

36. The method according to in claim 26, further comprising the step of using lipids as the coagulation-preventing structures (7) which are applied at least to the surfaces of the capsule units (4).

37. The method according to claim 36, further comprising the step of using phosphatidylcholine or other cell membrane constituents such as glycocalyx structures or glycocalyx components as the lipids.

38. The method according to claim 36, further comprising the step of binding the lipids (7) to proteins and/or glycolipids and/or glycoproteins.

39. The method according to claim 26, further comprising the step of using liver cells as the cells (5).

40. The method according to claim 26, further comprising the step of arranging the liver cells (5) bound in capsule units (4) in containers (1) provided with an inlet (2) and an outlet (3), and connecting the container (1) to the blood stream of a patient.

41. A capsule unit in which a multiplicity of cells (5) are arranged in an enveloping substance (6) surrounding the cells (5), the enveloping substance (6) being provided with coagulation-inhibiting agents of coagulation-preventing structures (7).

42. The capsule unit according to claim 41, wherein heparin is provided as the coagulation-inhibiting agent (7).

43. The capsule unit according to claim 41, wherein hirudin is provided as the coagulation-inhibiting agent (7).

44. The capsule unit according to claim 41, wherein prostaglandin is provided as the coagulation-inhibiting agent (7).

45. The capsule unit according to claim 41, wherein thromboxane structures are used as the coagulation-inhibiting agent (7).

46. The capsule unit according to claim 41, wherein polymers are provide as the enveloping substance (6).

47. A device for feeding living cells into a biological body fluid, particularly into a blood stream, with a container provided with and inlet (2) for the body fluid and with an outlet (3), a multiplicity of capsule units (4) being arranged in the container (1), which capsule units (4) are each formed by a plurality of cell (5) surrounded by an enveloping substance (6), the enveloping substance (6) being provided with coagulation-inhibiting agents or coagulation-preventing structures (7).

48. The device according to claim 47, wherein the container (1) is provided, in the area of the outlet (3), with a retention arrangement (8, 10) for the capsule units (4).

49. The device according to claim 48, wherein the retention arrangement (8) is a filter arrangement.

50. The device according to claim 48, wherein magnet parts or magnetizable substances (9) are embedded in the capsule units (4), and the retention arrangement (8) is provided with a magnetic separator (10).
